(19) United States
(12) Patent Application Publication
(10) Pub. No.: US 2004/0128167 A1
(43) Pub. Date: Jul. 1, 2004

(54) Main board with health inspection function and a health inspection system therewith

(76) Inventor: Tse-Hsien Liao, Taipei Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/722,493

(22) Filed: Nov. 28, 2003

(30) Foreign Application Priority Data
Dec. 2, 2002 (TW) 091134959

(51) Int. Cl. G06F 17/60; A61B 5/00

(52) U.S. Cl. 705/2; 600/300

(57) ABSTRACT

A health inspection system is provided. The health inspection system performs the functions of living body measurement and the integration as well as analysis of at least one health measurement device by means of a main board of a computer system directly, mainly comprising a signal processor, a register, a comparator, an alarm, a graph processor, and a sample data handler, additionally provided in the main board of the computer system. With the signal processor and the register, specific health signals transmitted from the plurality of health measurement devices may be processed simultaneously, and then integrated as well as analyzed by a central processing unit originally provided in the main board, graphed by the graph processor, compared by the sample data handler and the comparator, for warning or long-term storing the health measurement data with respect to living bodies of different identities. In this manner, not only various specific health signals may be integrated for providing an overall and accurate analysis with respect to health index, but also living bodies of different identities may be traced over a long period of time.

### FIELD OF THE INVENTION

The present invention is related to a health inspection system, particularly to a health inspection system performing the functions of living body measurement and the integration as well as analysis of at least one health measurement device by means of a main board of a computer system directly.

### BACKGROUND

Accordingly, due to the information revolution and urbanization development for the society, space and time for people to exercise is subject to invisible squeeze, such that the disease of civilization, for instance, hypertension, adiposity, diabetes, and heart rate variability, etc., also generally attack the modem inseparably. Thereby, it is extremely important for the modern to inspect various health indexes, such as high blood pressure, low blood pressure, body weight, body fat, heartbeat and sphygmus, blood sugar, etc., as examples, any time. Thus, numerous simple health measurement devices, such as sphygmomanometer, clinical thermometer, body fat meter, body weight scale, body height meter, blood sugar testing instrument, and electrocardiograph, as examples, are also rife in commercially available health care products.

Referring to FIG. 1, there is shown a conventional sphygmomanometer, mainly comprising a host 11, a display window 13, function keys 15, a signal transmission line 17, and a cuff with bladder 19. The cuff with bladder 19 is used to contact a part of body of the user in order for measuring specific health signals of the user, such as blood pressure and heartbeat, as examples. Next, this data may be processed initially to form a specific health signal enabling to be transmitted, and then, the latter may be delivered to the host 11 via the signal transmission line 17 and further processed by a device, such as microprocessor, provided inside the host. Finally, this signal may be displayed on the display window 13 for achieving the object of inspecting this specific health signal.

In the conventional health measurement device, however, there may exist disadvantages as follows:

1. For the conventional health measurement device, only specific health signals could be measured in accordance with the design thereof, and it is hardly for general public to integrate as well as analyze, and judge these specific health signals. In this case, overall as well as entire information with respect to health index could not be provided for the user.

2. A host and a display window may be both provided in the conventional health measurement, while the operation capability and the display image thereof may be limited by the design concept of light weight as well as handiness for the device itself, correspondingly. As a consequence, it is inconvenient for the user to select function and inspect measurement data.

3. In the conventional health measurement device, too many signal data could not be stored. As such, it is hardly to trace and inspect the variation in the health of the user over a long period of time, such that the specific health signal measured each time or in a short term may not be accurate objectively.

4. Limited by a poor capability for operation, health measurement data originating from a plurality of different health measurement devices or users could not be processed simultaneously.

### SUMMARY OF THE INVENTION

Thereby, in view of aforementioned disadvantages of the conventional health measurement system, how to provide an improved device with novelty and effect for effectively enhancing the convenience and humanization is the key point of the present invention.

Accordingly, it is a primary object of the present invention to provide a health inspection system directly utilizing a computer system connected to at least one health measurement device. Due to the high-speed operation, large memory size, and large-sized display image provided by the computer system itself, the effect of convenience and humanization to integrate various health measurement devices may be obtained.

It is a secondary object of the present invention to provide a health inspection system capable of collecting and analyzing specific health signal data originated from users of different identities in order to achieve an effect of expanding application field of the health measurement device.

It is another object of the present invention to provide a health inspection system allowed for simultaneously processing specific health signals inputted from various different health measurement devices, whereby the function of comprehensive decision and analysis for overall health indexes of the user may be obtained.

It is still another object of the present invention to provide a health inspection system capable of transmitting the result of health measurement of the user to a specific workstation, such as family doctor, for example, by means of network any time. Thereby, it is attainable to provide the health data for a remote doctor any time as well as over a long period of time, and the possibility of accident occurrence may be thus decreased effectively.

It is yet another object of the present invention to provide a main board with health inspection function for fulfilling aforementioned objects and effects.

For the purpose of achieving aforementioned objects, the main structure of a health inspection system of the present invention comprises: a main board having a central processing unit provided therein, the central processing unit being electrically connected to at least one signal receiver, peripheral device connecter, memory, signal processor, register, comparator, decoder, sample data handler, graph processor, and alarm, while the peripheral device connector being connected to a display and a data inputting means via a data transmission line; and a plurality of health measurement devices, each measuring a specific health signal of a living body, having a function processor and at least one signal transmitter provided therein, the specific health signal being transmitted to the signal receiver of the main board by the signal transmitter via a corresponding signal transmission line, and then integrated as well as analyzed by the main board to be stored in one of the memory, the sample data handler, and the combination thereof.

Additionally, a main board with health inspection function of the present invention comprises a central processing unit, and at least one signal receiver, peripheral device connecter, memory, and accessory means electrically connected to the central processing unit, respectively, wherein the accessory means comprises: a signal processor used for processing a specific health signal of a living body measured by at least one health measurement device and transmitted from the peripheral device connector; a register allowed for temporarily storing the specific health signal transmitted from the peripheral device connector; a decoder used for performing the decryption function for the specific health signal; a graph processor used for graphing the specific health signal; a sample data handler used for storing at least one reference sample data with respect to health; a comparator allowed for comparing the specific health signal with a predetermined health value established in advance; an alarm used for expressing a warning signal; and a network transmission means allowed for transmitting the specific health signal to a specific workstation via a network.

### BRIEF FUNCTION DESCRIPTION OF DRAWINGS

FIG. 1 is a structural schematic view of a conventional blood pressure measurement device;

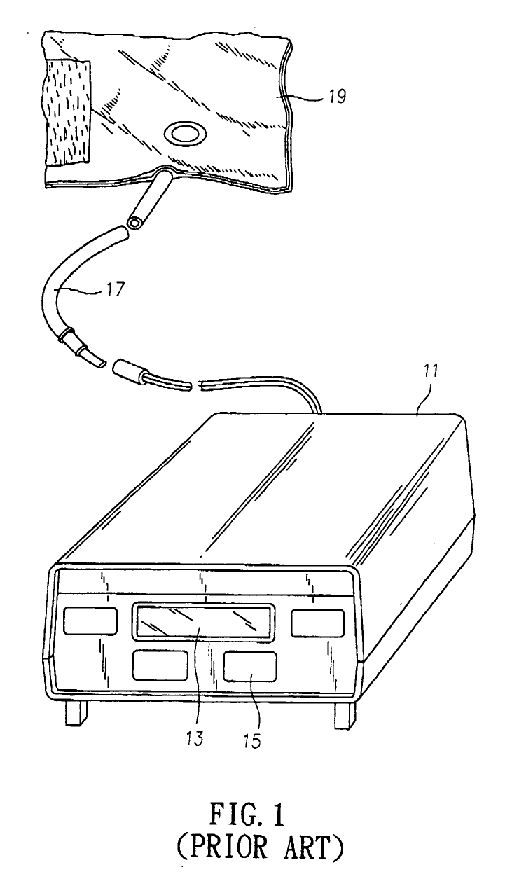

FIG. 2 is a schematic view of operation state according to one preferred embodiment of the present invention:

FIG. 3 is a schematic view of operation state according to another preferred embodiment of the present invention;

FIG. 4 is a diagram of functional configuration of a main board of the present invention; and

FIG. 5 is an operation flow chart of a health inspection system of the present invention.

### DETAILED DESCRIPTION

The structural features and the effects to be achieved may further be understood and appreciated by reference to the presently preferred embodiments together with the detailed description.

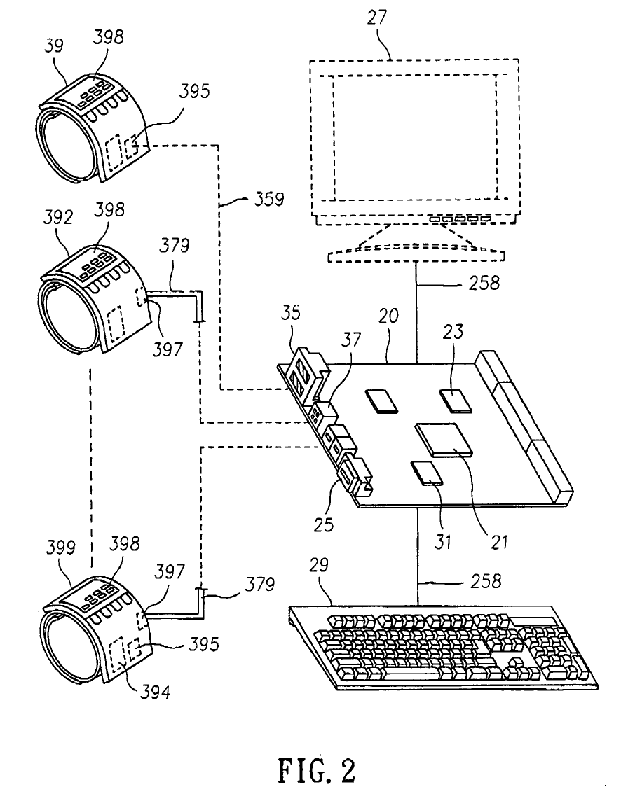

Firstly, referring to FIG. 2, there is shown a schematic view of operation state according to one preferred embodiment of the present invention. As shown in this figure, the present invention may be mainly applied to an existing computer system, with a main board 20 provided therein functioning as a processing center for receiving a specific health-signal from at least one health measurement device 39 to be operated. Except for a common central processing unit (CPU) 21, a memory 23, and a peripheral device connecter 25, an accessory means 31, a first signal receiver 35, and a second signal receiver 37 are further provided in the main board 20. In this embodiment, the first signal receiver 35 is presented as a wireless receiver, and the second signal receiver 37 is presented as a wired receiver.

For at least one health measurement device 39, 392, 399, such as the sphygmomanometer illustrated in this embodiment, as an example, only a cuff with bladder 19 commercially available, a brief function processor 394, provided therein, with a function of initial processing for health measurement data, for instance, transforming the health measurement data having been obtained into a health signal at specific frequency adapted for wireless transmission or wired transmission, and encrypting the measured data, etc., and at least one corresponding signal transmitter, for instance, a first transmitter (wireless transmitter) 395 and a second signal transmitter (wired transmitter) 397, as examples, are required, wherein the wireless transmitter 395 may be connected to the wireless receiver 35 on the main board 20 via a wireless transmission line 359, while the wired transmitter 397 may be electrically connected to the wired receiver 37 on the main board 20 via a wired transmission line 379.

As a health measurement data is measured by the sphygmomanometer 39, 391, or 399 allowed for contacting a user of living body, the data is processed by the initial data processing provided by the brief function processor 394, and then transmitted by the use of transmission capability of the wireless transmitter 395, such that the specific health signal is transmitted to the main board 20 in the form of a wireless transmission signal 359 and through the wireless receiver 35, or transmitted to that main board through the transmission path composed of the wired transmitter 397. wired transmission line 379, and wired receiver 20, and is further operated or controlled by the central processing unit 21 as well as the accessory means 31 provided in the main board 20. The specific health signal data, having been processed and operated, may be thus stored in the memory 23, or displayed on a display 27 of larger display size via the peripheral device connecter 25 or a data transmission line 258. In this manner, not only a convenient inspection for the measurement result is provided for the user, but also a powerful computer system is utilized. As such, the purpose of tracing for health care over a long period of time may be obtained.

The main board 20 of that computer system, of course, may be also connected to a data inputting means 29, such as a keyboard, for example, via the peripheral device connecter 25 or data transmission line 258. A specific cooperating data, such as the identity data of a user, may be inputted from the data inputting means in order for classifiedly inspecting or storing the specific health signal having been examined or measured. In the present invention, only one main board 20 is necessary for receiving health measurement from different sphygmomanometers or users 39, 391, 399 simultaneously. Moreover, the sphygmomanometer 39 may be further provided with a smaller display window showing the measurement result and other function keys 398. The function keys 398 may also comprise a function of inputting the identity data.

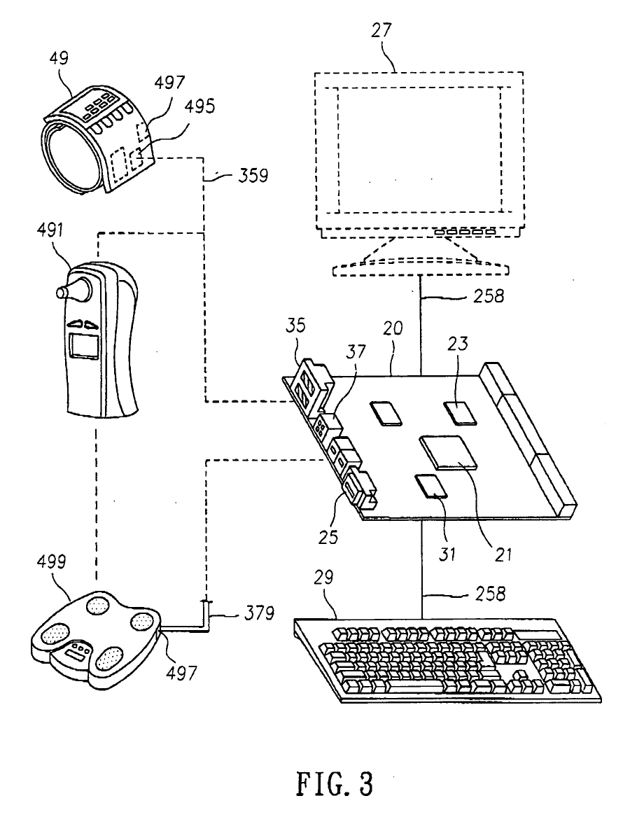

Furthermore, referring to FIG. 3, there is shown a schematic view of operation state according to another embodiment of the present invention. In this embodiment, the main board 20 is still capable of receiving at least one health measurement device, such as sphygmomanometer 49, clinical thermometer 491, body weight scale 499, or body fat meter, body height scale, blood sugar testing instrument, electrocardiograph, etc., as examples. Each of the specific health signals from each health measurement device 49, 491, 499 may be collected for the main board 20 by means of the designed wireless transmitter 495 or wired transmitter 497. In this manner, instead of inspecting only one single item of health measurement as the conventional device, a comprehensive decision for general health index of the user is obtained owing to the powerful main board 20 in the computer system. Therefore, a much more overall, convenient, accurate, and humanized function of the health measurement may be provided for the user.

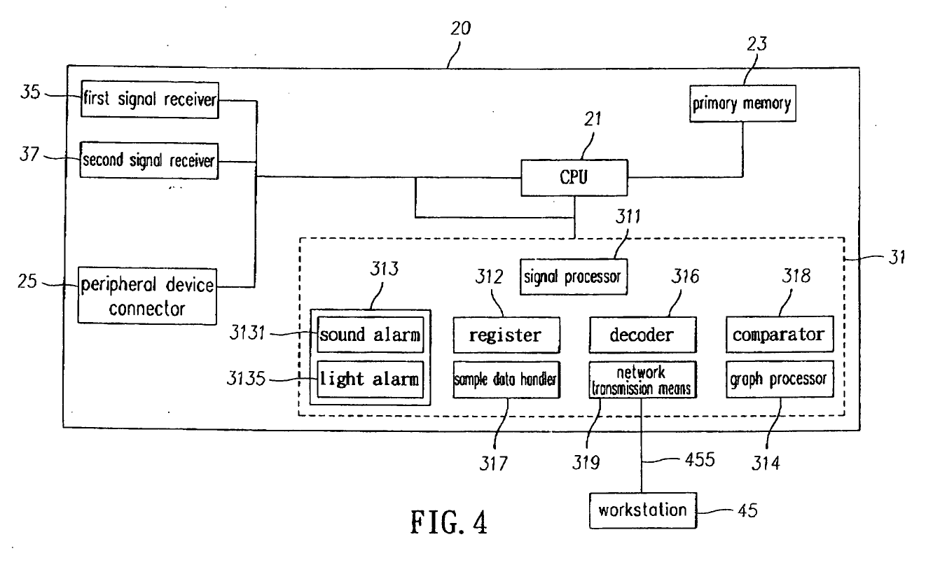

Additionally, referring to FIG. 4, there is shown a diagram of functional configuration of the main board according to the present invention. As shown in this figure, except for the central processing unit 21, memory 23, first signal receiver (wireless receiver) 35, second signal receiver (wired receiver) 37, and peripheral device connecter 25, the main board 20 in this embodiment may be mainly provided with the accessory means 31. The accessory means 31 mainly comprises a signal processor 311 allowed for receiving the specific health signal transmitted from the wired receiver 35 or wireless receiver 37, and for initially processing that signal, such as transforming the inputted signal into a digital signal, etc., for example.

The inputted signal waiting for being processed may be stored in a register 312 in advance, if the specific health signals inputted simultaneously include a plurality of different groups, or if another program is executed by the central processing unit 21 in the meantime. Furthermore, if the inputted health measurement signal is encrypted, or needs the recognition of identity, a decoder 316 is required for decrypting.

Furthermore, the specific health signal having been inputted and processed may be compared with a predetermined health value established in a comparator 318 in advance. If the measured specific health signal is greater than one predetermined health value, an alarm 313, such as a sound alarm 3131 or a light alarm 3135, is used for emitting sound or light in order to warn the user what health item should be paid attention to.

A data storage device with larger memory size must be used, due to the fact that not only the obtained data is large, owing to the capability of simultaneously receiving measurement signals from different health measurement devises, or receiving each specific health signal from different users, but also that data should be stored over a long period of time to be traced. In this case, the memory 23 in the computer system is a preferred choice.

Furthermore, difference in the health measurement data may be possibly resulted from different health statuses of the user, or different measurement time. Thereby, an individual reference sample data is required for a long-term tracing, or for individual measurement at each time, in order to provide a more objective comparison. In the present invention, thus, a sample data handler 317 allowed for storing each reference sample data may be also provided in the main board 20. In another embodiment, however, the health measurement signal of the user or predetermined health value may be also stored in the sample data handler 317.

Further, for the purpose of providing the user with a preferred display image or hint when inspecting the health measurement signal. All the inputted or stored health measurement signals may be processed by a graph processor 314, in such a way that the humanized display image as well as easy manipulation may be obtained.

Furthermore, for the sake of instantaneously watching or providing for the remote medical personnel with the specific health signal of the user as reference, the signal data may be transmitted through the peripheral device connecter 25 or a network transmission means 319, and then via a network 455 to a specific workstation 45, such as hospital, rest home, and medical station, as examples.

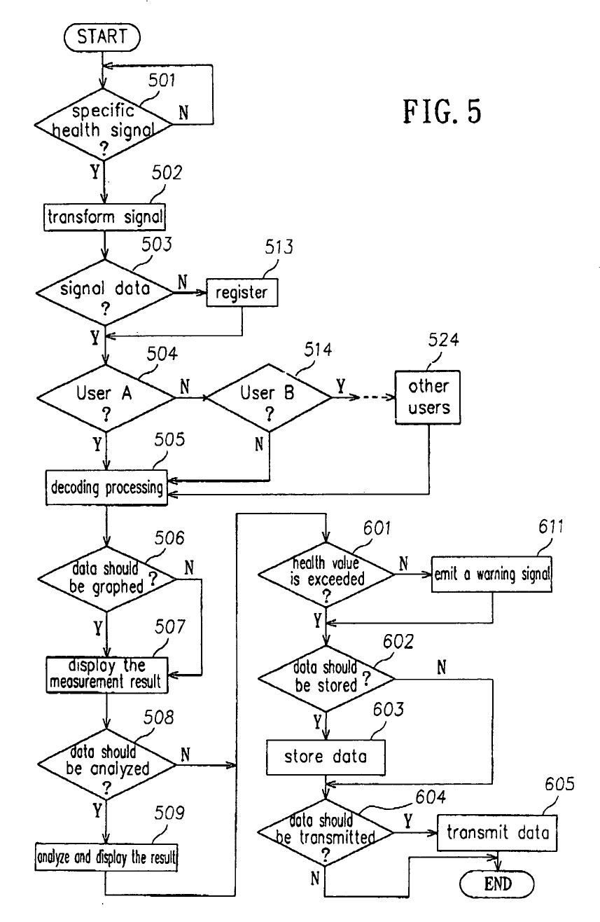

Finally, referring to FIG. 5, there is shown an operation flow chart of the health inspection system according to the present invention. As illustrated in this figure, combining with FIG. 3, the operation of the main board of the present invention comprises the steps as follows:

In step 501, the main board 20 may monitor whether any one specific health signal is inputted via the wireless receiver 35 or wired receiver 37 any time. If so, keep waiting; while if not, go to step 502.

In step 502, the inputted signal is processed initially by the signal processor 311.

In step 503, the signal processor 311 may determine whether the inputted signal is one single data or not. If not, the inputted data waiting for being processed is previously stored in the register 312; while if so, the procedure may proceed to step 504.

In step 504, one user of living body, whom the specific health signal belongs to, may be determined by the decoder 316 or data inputting means (29). If that user of living body is user A, the procedure may proceed to step 505; while if it is not user A, the procedure may proceed to determine whether that user of living body is user B or not. If so, the procedure may proceed Mu stop 505; while if not, the procedure may continue to determine or recognize the identity of the user. If there is none of the filed users found, that body of living body is categorized as other users, stop 524.

In step 505, as the dependence on the identity of the user exists, or the specific health signal is encrypted, a decoding operation is performed by the decoder 316.

In step 506, it is determined whether the inputted data should be graphed by the graph processor 314, and the corresponding measurement result based on the selected message should be displayed on the monitor (27), as described in step 507.

In step 508, inquiring whether a comprehensive or long-term integration as well as analysis with the previously stored specific health signal, or other different specific signals is required. If so, the procedure may proceed to step 509; if not, it may go to step 601.

In step 509, the result based on the selected item is displayed on the monitor (27).

In step 601, whether the specific health signal is greater than a predetermined health value previously established may be perceived depending on the comparison performed by a comparator 318. If so, a warning message is emitted by the alarm 313 to warn the user, as described in step 611; if not, the procedure may proceed to step 602.

In step 602, inquiring whether the data, such as the specific health signal measured this time, should be stored in a corresponding file depending on the difference in users, or the difference in specific health signals. If so, the procedure may proceed to step 603; if not, the procedure may go to step 604.

In step 603, the data, such as the specific health signal measured this time, may be stored in the memory 23 or sample data handler 317.

In step 604, inquiring whether the data should be transmitted to the remote specific workstation 45 to be explained by the medical personnel. If so, the procedure may proceed to step 605; if not, this operation will be ended.

Moreover, in step 605, the data, such as the specific health signal, etc., is transmitted to one specific workstation 45 by means of the network transmission means 319, peripheral device connecter 25, and network 455.

Although a main board in the computer system is considered as an illustrative example in the aforementioned embodiment, however, the main board may be also effectively applied to a personal computer, notebook, personal digital assistant (PDA), and mobile phone.

To sum up, the present invention is related to a health inspection system, particularly to a health inspection system performing the functions of living body measurement and the integration as well as analysis of at least one health measurement device by means of a main board of a computer system directly. Therefore, this application is filed in accordance with the patent law duly, since the present invention is truly an invention with novelty, advancement or non-obviousness, and availability by the industry, thus naturally satisfying the requirements of patentability. Your favorable consideration will be appreciated.

The foregoing description is merely one embodiment of present invention and not considered as restrictive. All equivalent variations and modifications in process, method, feature, and spirit in accordance with the appended claims may be made in any way from the scope of the invention.

### LIST OF REFERENCE SYMBOLS

11 host

13 display window

15 function key

17 signal transmission line

19 cuff with bladder

20 main board

21 central processing unit

23 memory

25 peripheral device connecter

258 data transmission line

27 display

29 data inputting means

311 signal processor

312 register

313 alarm

3131 sound alarm

3135 light alarm

314 graph processor

316 decoder

317 sample data handler

318 comparator

319 network transmission means

35 first signal receiver

359 first signal transmission line

37 second signal receiver

379 second signal transmission line

39 health measurement device

392 health measurement device

394 brief function processor

395 first signal transmitter

397 second signal transmitter

398 function key and display window

399 health measurement device

45 specific workstation

455 network

49 sphygmomanometer

491 clinical thermometer

495 first signal transmitter

497 second signal transmitter

499 body weight scale

1. A health inspection system, comprising:
a main board having a central processing unit provided therein, said central processing unit electrically connected with at least one signal receiver, peripheral device connecter, memory, signal processor, register, comparator, decoder, sample data handler, graph processor, and alarm, said peripheral device connector connected with a display and a data inputting means through a data transmission line; and
a plurality of health measurement devices, each measuring a specific health signal of a living body, having a function processor and at least one signal transmitter provided therein, said specific health signal transmitted to said signal receiver by said signal transmitter through a corresponding signal transmission line;
wherein after integrating and analyzing by said main board, said specific health signal is stored in one of said memory, said sample data handler, or said combination thereof.

2. The health inspection system according to claim 1, wherein said data inputting means is used for inputting the identity data of said living body, said specific health signal of each measurement stored in a corresponding specific file selected from the group consisting of said memory, said sample data handler, or the combination thereof.

3. The health inspection system according to claim 1, wherein said health measurement device is selected from the group consisting of a sphygmomanometer, clinical thermometer, body fat meter, body weight scale, body height meter, blood sugar testing instrument, electrocardiograph, or the combination thereof.

4. A health inspection system, comprising:
a main board having a central processing unit provided therein, said central processing unit electrically connected to at least one signal receiver, peripheral device connecter, memory, and accessory means, said peripheral device connector being connected to a display through a data transmission line; and
at least one health measurement devices, measuring a specific health signal of a living body, having a function processor and at least one signal transmitter provided therein, said specific health signal being transmitted to said signal receiver of said main board by said signal transmitter through a corresponding signal transmission line.

5. The health inspection system according to claim 4, wherein said accessory means of said main board is selected from the group consisting of a signal processor, register, alarm, graph processor, decoder, sample data handler, comparator, network transmission means, or the combination thereof.

6. The health inspection system according to claim 5, wherein said decoder is provided for recognizing the identity data of said measured living body.

7. The health inspection system according to claim 4, wherein said signal receiver of said main board is selected from the group consisting of a wired signal receiver, wireless signal receiver, or the combination thereof.

8. The health inspection system according to claim 4, wherein said health measurement device is selected from the group consisting of a sphygmomanometer, clinical thermometer, body fat meter, body weight scale, body height meter, blood sugar testing instrument, electrocardiograph, or the combination thereof.

9. The health inspection system according to claim 5, wherein said alarm is selected from the group consisting of a sound alarm, light alarm, or the combination thereof.

10. The health inspection system according to claim 4, further comprising a data inputting means connected to said peripheral device connecter of said main board through a data transmission line.

11. The health inspection system according to claim 4, wherein the application field of said main board is selected from the group consisting of a personal computer, notebook, personal digital assistant device, mobile phone, or the combination thereof.

12. The health inspection system according to claim 4, wherein said signal transmission line of said health measurement device is selected from the group consisting of a wired line, wireless line, or the combination thereof.

13. The health inspection system according to claim 5, wherein said network transmission means is provided for transmitting said specific health signal to a specific workstation through a network.

14. A main board with health inspection function mainly comprises a central processing unit, at least one signal receiver, peripheral device connecter, memory, signal processor, comparator, and alarm, wherein said signal receiver receives a specific health signal of living body, measured by at least one health measurement device through a signal transmission line, said specific health signal of living body being stored in said memory after operated by said central processing unit and said signal processor, said specific health signal transmitted by said peripheral device connector to a display through a data transmission line.

15. The main board according to claim 14, further comprising a graph processor provided for graphing said specific health signal.

16. The main board according to claim 14, wherein said peripheral device connecter is further connected to a data inputting means through a data transmission line, said data inputting ,means provided for inputting the identity data of said living body.

17. The main board according to claim 14, further comprising a decoder capable of recognizing the identity data of said health measurement device.

18. The main board according to claim 14, further comprising a register capable of temporarily storing the signal data transmitted originated from said signal transmission means.

19. The main board according to claim 14, further comprising a sample data handler provided for storing at least one reference sample data with respect to health,

20. The main board according to claim 14, wherein said comparator is further provided with a predetermined health value therein.

21. The main board according to claim 14, wherein said alarm is selected from the group consisting of a sound alarm, light alarm, or the combination thereof.

22. The main board according to claim 14, further comprising a network transmission means provided for transmitting said specific health signal to a specific workstation.

23. The main board according to claim 14, wherein said health measurement device is selected from the group consisting of a sphygmomanometer, clinical thermometer, body fat meter, body weight scale, body height meter, blood sugar testing instrument, electrocardiograph, Or the combination thereof.

24. The main board according to claim 14, wherein the application field of said main board is selected from the group consisting of a personal computer, notebook, personal digital assistant device, mobile phone, or the combination thereof.

25. A main board with health inspection function comprises a central processing unit, and at least one signal receiver, peripheral device connecter, memory, and accessory means electrically connected to said central processing unit, respectively, wherein said accessory means comprises:
a signal processor used for processing a specific health signal of a living body measured by at least one health measurement device and transmitted from said peripheral device connecter,
a register allowed for temporarily storing said specific health signal transmitted from said peripheral device connecter;
a decoder used for performing the decryption function for said specific health signal;
a graph processor used for graphing said specific health signal;
a sample data handler used for storing at least one reference sample data with respect to health;
a comparator allowed for comparing said specific health signal with a predetermined health value established in advance;
an alarm used for expressing a warning signal; and
a network transmission means allowed for transmitting said specific health signal to a specific workstation via a network.

26. The main board according to claim 25, wherein said alarm is selected from the group consisting of a sound alarm, light alarm, or the combination thereof.

27. The main board according to claim 25, wherein said health measurement device is selected from the group consisting of a sphygmomanometer, clinical thermometer, body fat meter, body weight scale, body height meter, blood sugar testing instrument, electrocardiograph, or the combination thereof.
